(19) United States
(12) Patent Application Publication
(10) Pub. No.: US 2004/0241225 A1
(43) Pub. Date: Dec. 2, 2004

(54) Modulation of vitamin storage

(76) Inventors: Simon Michael West, Williamstown (AU); David Kannar, Belgrave South (AU)

Correspondence Address: Jules E Goldberg, Reed Smith, 29th Floor, 599 Park Avenue, New York, NY, 10022, US

(21) Appl. No.: 10/487,743

(22) PCT Filed: Sep. 26, 2002
(86) PCT No.: PCT/AU02/01321

(30) Foreign Application Priority Data
Sep. 26, 2001 (WO) PCT/AU01/01206
Aug. 27, 2002 (AU) 2002951045

(51) Int. Cl. A61K 31/355; A61K 9/20; A61K 31/12; A61K 31/07

(52) U.S. Cl. 424/465; 514/458; 514/690; 514/725

(57) ABSTRACT

A method for increasing levels of a storage form of a vitamin selected from the group consisting of tocopherol, retinol, vitamin K1 and mixtures thereof in a target tissue of a subject, the method comprising administering to the subject an effective amount of a phosphate derivative of the vitamin so as to cause an accumulation of stored vitamin in the target tissue.

### FIELD OF THE INVENTION

The invention relates to modulating vitamin levels in animals, particularly levels of endogenous storage forms of tocopherol (Vitamin E), retinol (Vitamin A) or Vitamin K1 by administration of phosphate derivatives.

### BACKGROUND OF THE INVENTION

In this specification, where a document, act or item of knowledge is referred to or discussed, this reference or discussion is not an admission that the document, act or item of knowledge or any combination thereof was at the priority date:

(a) part of common general knowledge; or

(b) known to be relevant to an attempt to solve any problem with which this specification is concerned.

Whilst the following discussion concerns tocopherol and tocopheryl phosphate (TP), it is to be understood that this is merely illustrative and that the invention is not limited to tocopherol or TP but that the invention also similarly relates to retinol and vitamin K1 and their storage and transport forms.

Vitamin E is widely recognised as an anti-oxidant of considerable biological importance. It is a potent free radical scavenger with a vital role in the maintenance of cellular integrity through its capacity to protect the polyunsaturated fatty acyl moieties of phospholipids in biological membranes and plasma lipoproteins. Vitamin E consists of the group of isoprenoids know as ‘tocopherols’ which provide benefits to the health and well-being of humans and animals. Several different tocopherols having Vitamin E activity are known, the most active and abundant being α-tocopherol.

There have been many attempts to supplement the body's dietary intake of vitamin E. In order for supplements of biologically active species and nutrients to be valuable in clinical practice, they should exhibit certain properties. For example, the supplements should have adequate stability, solubility, permeability and bioavailability.

Free tocopherol is not stable and therefore not suitable as a bioavailable delivery form of Vitamin E. For this reason, derivatisation has long been recognised as an important means of increasing stability and bioavailability of tocopherol, but it is generally accepted that absorption of these derivatives has still been low. Tocopherol organic esters, such as tocopheryl acetate or tocopheryl succinate are examples of such derivatised species which are currently in widespread commercial use. These esters are cheap to produce and more stable than free tocopherol.

In order to be useful, tocopherol derivatives should be capable of converting to the biologically active species after they have been absorbed into the systemic circulation of a human or animal subject. Formulators believed that the free form of tocopherol was biologically active and thus believed that when the pancreatic esterase and lipase activity released free tocopherol from the organic esters of tocopherol in the small intestine lumen, the body's levels of Vitamin E were increased.

When investigating the effectiveness of various tocopherol derivatives as sources of tocopherol as dietary supplements, analytical techniques of the prior art have concentrated on measuring how efficiently these supplements increase levels of free tocopherol in plasma and tissue. Tocopherol acetate has been favoured as a Vitamin E dietary supplement of the prior art because it reportedly causes an immediate, readily measurable increase in plasma free tocopherol levels.

Formulators who have been attempting to increase the water solubility of tocopherol derivatives have prepared formulations containing TP as it is much more water soluble than organic esters of tocopherol. While TP has been used as a water soluble source of Vitamin E in vitro, it has not previously been commonly used as a suitable in vivo source of Vitamin E because the phosphate group is a substrate of phosphorylases, is resistant to passive transport, and therefore considered to interfere with absorption.

To date, Vitamin E supplements containing either tocopheryl acetate or tocopheryl phosphate have aimed to provide a recommended daily intake of 7-15 mg/day per person of free tocopherol in the body. Supplements have contained 200-600 mg as this amount is considered to be innocuous in adult humans, although there is limited evidence of this.

Paradoxically, as well as being a potent free radical scavenger α-tocopherol is also a pro-oxidant and a potential source of free radicals which would cause damage to the body. Free tocopherol is pro-oxidant at high concentrations so it is unlikely that this is the biological storage or transport form within the body. Being fundamentally important to membrane stability, the natural form must be transported efficiently and mobilized on demand to act as an electron transfer agent. To date only free and protein-bound or associated α-tocopherol have been detected in biological tissue. The protein-bound and associated forms are thought to represent transport forms. Although it would appear to be a vital piece of information, no one has pursued the concept that although free tocopherol is the biologically active form there must be another form which is stored or transported through the body. All attempts at supplementation of Vitamin E have focussed on increasing the levels of free tocopherol within tissue or plasma. There have been no attempts to increase the storage or transport form of Vitamin E.

These negative effects of high levels of free tocopherol limit the daily recommended dose. There is also a concern that a high intake of fat soluble nutrients can lead to toxicity. As a result, the recommended daily intake has limited the amount of tocopherol provided by supplements.

### SUMMARY OF THE INVENTION

It has now been found by the present inventors that the storage form of tocopherol, retinol and vitamin K1 is their respective phosphate derivatives. These phosphate derivatives are not typically a source of free radicals. Surprisingly, the present inventors have found that administering phosphate derivatives of tocopherol, retinol or vitamin K1 to an animal can provide therapeutically useful levels of tocopherol, retinol or vitamin K1 in storage and transport forms.

Accordingly, in a first aspect, the present invention provides a method for increasing levels of a storage form of a vitamin selected from the group consisting of tocopherol, retinol, vitamin K1 and mixtures thereof in a target tissue of a subject, the method comprising administering to the subject an effective amount of a phosphate derivative of the vitamin so as to cause an accumulation of stored vitamin in the target tissue.

Preferably, the vitamin is tocopherol.

Preferably, the subject is an animal, more preferably the animal is a human.

Preferably, the target tissue is liver because it is the storage organ of preference and the generation site of lipoprotein cholesterol. It is also known that there is Vitamin E activity in the skin and TP is important in skin metabolism. Vitamin E may also be important for adipose tissue where it may protect against degradation of unsaturated fats. Vitamin E may also be important in the brain.

Preferably, the increased levels of the stored vitamin(s) result in potentially therapeutic levels of the vitamin(s) in the animal.

The present invention is particularly suitable for increasing the storage levels of the vitamins to be of therapeutic value. Increased levels of vitamins, particularly tocopherol, can have uses in the treatment of inflammatory diseases such as, but not limited to, coronary diseases, atherosclerosis and diabetes and a number of diseases affected by tocopherol, such as cancer where tocopherol affects cell adhesion, and foetal development. In the brain, vitamin levels may assist treatment of alzheimers.

Therapeutic levels of the vitamins would typically be in the order of at least about 50% increase of the levels usually stored in healthy or normal tissue. Preferably, the levels are increased by at least about 100%, more preferably at least about 200%, that is, about 2 to 3 times normal plasma or tissue levels. By ‘loading up the body’ with the vitamin using the present invention, higher levels of vitamins can then be released in a number of ways to provide potentially higher doses of biologically available vitamin to organs or tissue in the body as required.

The present invention may also be suitable for treating or overcoming problems with subjects unable to acquire or utilise vitamins by normal dietary uptake or processing. The ability to increase storage levels may also be useful to overcome consequences of periods of low or poor dietary intake.

Tocopherol is used in three forms in the body, being

| | | Typical levels in normal |
|---|---|---|
| Form of tocopherol | | adult human |
| (a) | ‘free’ as described by the current | total plasma tocopherols |
| | analytical methods where it is readily | range from about |
| | able to be oxidized to the quinone form | 5 to 12 μg/ml |
| | or take part in free radical reactions; | |
| (b) | present as TP in unique places such as | This amount has not been |
| | the boundary between the epidermis | quantified in the literature |
| | and dermis where tocopherol acts in the | |
| | initiator of the inflammatory response | |
| | and other cell mediation; | |
| (c) | storage of tocopherol in an inactive | human adipose |
| | form. | free tocopherol - |
| | | 25 ± 9 μg/gm, |
| | | TP - Adipose |
| | | 24 ± 9 μg/gm & |
| | | Liver 32 ± 2 μg/gm |

Typically, the increased storage forms acts as a source of tocopherol, retinol or vitamin K1 to tissue, organs or cells of the subject.

The present inventors have found that at least about 3 mg/kg (at least about 240 mg in an 80 kg adult), typically from about 3 to 50 mg/kg of the phosphate derivative of the vitamin is required to increase storage levels to a beneficial amount. More preferably, 3 to 30 mg/kg is provided. More preferably, at least 10 mg/kg body mass of tocopherol provided orally as tocopheryl phosphate has been found suitable to achieve a level of 68% absolute bioavailability. This is significantly more than the recommended daily intake (RDI) of 7-15 mg/day for a normal person.

The dosage may be achieved by administering the phosphate derivative of the vitamin(s) in one dose or may be administered over a period of minutes, hours or days to achieve the required stored amount of the vitamin(s).

The phosphate derivative of the vitamin can be given by any suitable route such as orally or parenterally.

In a second aspect, the present invention provides a method for alleviating or treating a subject suffering a condition responsive to a vitamin treatment, the method comprising administering to a subject in need of such treatment an amount of a phosphate derivative of the vitamin selected from the group consisting of tocopherol, retinol, vitamin K1 and mixtures thereof effective to cause an accumulation of a therapeutic amount of tocopherol, retinol vitamin K1 or a mixture thereof in a tissue of the subject.

Preferably, the conditions expected to be responsive to a vitamin treatment are inflammatory diseases such as, but not limited to, coronary diseases, artherosclerosis and diabetes and a number of diseases affected by tocopherol, such as cancer where tocopherol affects cell adhesion, and foetal development.

Preferably, the vitamin is tocopherol.

Preferably, the subject is an animal, more preferably the animal is a human.

Preferably, the target tissue is liver as this tissue can be used to indicate that there are adequate stores for the various ubiquitous metabolic activities in different tissues.

The phosphate derivative of the vitamin can be given by any suitable route such as orally or parenterally.

The present inventors have found that at least about 3 mg/kg (at least about 240 mg in an 80 kg adult), typically from about 3 to 30 mg/kg of the phosphate derivative of the vitamin is required to increase storage levels to a beneficial amount which can result in therapeutic levels of the vitamin(s). More preferably, about 10 mg/kg body mass of tocopherol provided orally as tocopheryl phosphate has been found suitable to achieve a level of 68% absolute bioavailability. This is significantly more than the recommended daily intake (RDI) of 7-10 mg/day for a normal person.

In a third aspect, the present invention provides use of an effective amount of a phosphate derivative of a vitamin selected from the group consisting of tocopherol, retinol, vitamin K1 and mixtures thereof in the manufacture of a supplement for causing an accumulation of stored vitamin in the target tissue of an animal.

In a fourth aspect, the present invention provides use of an effective amount of a phosphate derivative of a vitamin selected from the group consisting of tocopherol, retinol, vitamin K1 and mixtures thereof in the manufacture of a medicament for alleviating or treating a subject suffering a condition responsive to a vitamin treatment.

The term “phosphate derivatives of the vitamin” is used herein to refer to the acid forms of phosphorylated tocopherol, retinol or vitamin K1, salts of the phosphates including metal salts such as sodium, magnesium, potassium and calcium and any other derivative where the phosphate proton is replaced by other substituents such as ethyl or methyl groups or phosphatidyl groups.

In some situations, it may be necessary to use a phosphate derivative such as a phosphatide where additional properties such as increased water solubility are preferred. Phosphatidyl derivatives are amino alkyl derivatives of organic phosphates. These derivatives may be prepared from amines having a structure of R₁R₂N(CH₂)ₙOH wherein n is an integer between 1 and 6 and R₁ and R₂ may be either H or short alkyl chains with 3 or less carbons. R₁ and R₂ may be the same or different. The phosphatidyl derivatives are prepared by displacing the hydroxyl proton of the electron transfer agent with a phosphate entity that is then reacted with an amine, such as ethanolamine or N,N′ dimethylethanolamine, to generate the phosphatidyl derivative of the electron transfer agent. One method of preparation of the phosphatidyl derivatives uses a basic solvent such as pyridine or triethylamine with phosphorous oxychloride to prepare the intermediate which is then reacted with the hydroxy group of the amine to produce the corresponding phosphatidyl derivative, such as P cholyl P tocopheryl dihydrogen phosphate.

In some situations complexes of phosphate derivatives of the vitamins may also be utilized where additional properties such as improved stability or deliverability may be useful. The term “complexes of phosphate derivatives of the vitamin” refers to the reaction product of one or more phosphate derivatives of tocopherol, retinol or vitamin K1 and mixtures thereof with one or more complexing agents selected from the group consisting of amphoteric surfactants, cationic surfactants, amino acids having nitrogen functional groups and proteins rich in these amino acids as disclosed in international patent application no PCT/AU01/01476.

The preferred complexing agents are selected from the group consisting of arginine, lysine and tertiary substituted amines, such as those according to the following formula:

NR¹R²R³

wherein R¹ is chosen from the group comprising straight or branched chain mixed alkyl radicals from C6 to C22 and carbonyl derivatives thereof;

R² and R³ are chosen independently from the group comprising H, CH₂COOX, CH₂CHOHCH₂SO₃X, CH₂CHOHCH₂OPO₃X, CH₂CH₂COOX, CH₂COOX, CH₂CH₂CHOHCH₂SO₃X or CH₂CH₂CHOHCH₂OPO₃X and X is H, Na, K or alkanolamine provided R² and R³ are not both H; and

wherein when R¹ is RCO then R² may be CH₃ and R³ may be (CH₂CH₂)N(C₂H₄OH)—H₂CHOPO₃ or R² and R³ together may be N(CH₂)₂N(C₂H₄OH)CH₂COO—.

Preferred complexing agents include arginine, lysine or lauryliminodipropionic acid where complexation occurs between the alkaline nitrogen center and the phosphoric acid ester to form a stable complex.

The administration of phosphate derivatives of the vitamin to provide a ‘therapeutic effect’ includes administration to achieve a curative, preventative or other beneficial health effect. For example, administration to a subject may be undertaken to treat a deficiency of the vitamin, to ameliorate or cure a disease or disorder, to ameliorate or remove the symptoms of the disease or disorder, or to increase the subject's plasma and tissue level of the vitamin to provide a beneficial effect.

The phosphate derivative of the vitamin may be administered to humans or animals through a variety of dose forms such as supplements, enteral feeds, parenteral dose forms, suppositories, nasal delivery forms, dermal delivery including patches, creams, and any other delivery system capable of supplementing the natural storage and transport form of the electron transfer agent.

For example the phosphate derivative of the vitamin may be administered by an orally or parenterally administered dose form. These include, tablets, powders, chewable tablets, capsules, oral suspensions, suspensions, emulsions or fluids, children's formulations, enteral feeds, nutraceuticals, and functional foods.

The dose form may further include any additives routinely used in preparation of that dose form such as starch or polymeric binders, sweeteners, coloring agents, emulsifiers, coatings and the like. Other suitable additives will be readily apparent to those skilled in the art.

In one embodiment, the dose form has an enteric coating as disclosed in international patent application PCT/AU01/01206.

In another embodiment, the dose form is a topical formulation as disclosed in international patent application PCT/AU02/01003.

The term “endogenous” refers to a vitamin occurring in the body as the result of ingestion from a diet with no supplementation of the vitamin or its derivatives, and where only typical metabolic processes transform the dietary vitamin.

In order that the present invention may be more clearly understood, preferred forms will be described with reference to the following drawings and examples.

### BRIEF DESCRIPTION OF DRAWINGS

The following figures are referred to in the examples.

FIG. 1: Calibration curve for TP vs T₂P using ESMS.

FIG. 2: GCMS of methylated liver extract and TP standard.

FIG. 3: Example of TP ESMS spectra.

FIG. 4: Example of liver extract ESMS spectra.

### MODES FOR CARRYING OUT THE INVENTION

It has now been demonstrated that the monophosphate ester of α-tocopherol, α-tocopheryl phosphate, is present in significant quantities (8-147 μg/g) in a range of biological tissues. α-Tocopheryl phosphate is resistant to oxidation and is thus inactive as an anti-oxidant; it is also resistant to acid and alkaline hydrolysis so that it cannot be detected by the standard assays for Vitamin E. The discovery of this storage form of Vitamin E heralds the need for a reassessment of the role of this essential vitamin in the body.

For example, without being bound by theory, it is believed that TP as a nascent storage form is not a source of free radicals in marked contrast to free tocopherol which is an excellent source of undesirable free radicals or pro-oxidant at high concentrations. Oxidation with active phosphorylation of adenosine diphosphate for example, is known suggesting that α-tocopheryl phosphate may be a reducing agent capable of producing phosphorylated secondary messengers. This would account for the non-antioxidant roles ascribed to tocopherol.

Again without wishing to be bound by theory, the following model is proposed to help understand the role of α-tocopheryl phosphate in tissue. α-Tocopheryl phosphate is stored in the lipoprotein environment in association with a lipophilic protein or as a glycerophosphate derivative. The proximity of a free radical or the generation of an oxidative environment stimulates the dephosphorylation of α-tocopheryl phosphate and the release of free α-tocopherol. When the oxidative challenge is neutralised, excess antioxidant, (α-tocopherol) is drawn back into storage by phosphorylation. This conservative strategy avoids pro-oxidation. This proposed mode of action for α-tocopheryl phosphate may also apply to other lipophilic bioactive compounds and provide a new explanation for its function in vivo.

The invention will now be further illustrated and explained in the following non-limiting examples.

### EXAMPLES

### Example 1

In this example, the storage form of Vitamin E was investigated in various tissues.

The standard analytical methodology for the detection of α-tocopherol in tissue samples and foodstuffs normally includes a hydrolysis step as part of the extraction process. The hydrolysis ensures that α-tocopheryl esters, such as added α-tocopherol acetate, are converted to free α-tocopherol prior to analysis.

There is scant literature on the analysis of α-tocopheryl phosphate. The earliest report appears to be a method for the separation of synthetic α-tocopheryl phosphate from other phosphate esters using paper chromatography. Although the authors reported good sensitivity, they failed to detect natural levels of α-tocopheryl phosphate in rat liver. It seems highly likely that the α-tocopheryl phosphate was lost at the step in their protocol at which protein was removed from the homogenate.

Surprisingly, α-tocopheryl phosphate is resistant to all of the alkaline and acid hydrolysis conditions encountered in these procedures and is consequently not included with free α-tocopherol in typical analyses. Indeed, refluxing α-tocopheryl phosphate under strongly alkaline or acidic conditions for extended periods in excess of 24 hours does not lead to any cleavage of the phosphate bond. We have been unable to discover conditions under which α-tocopheryl phosphate can be hydrolysed to yield free α-tocopherol. α-Tocopheryl phosphate has also proven to be resistant to oxidation and does not give a positive colour test under conditions of the standard colorimetric α-tocopherol analysis.

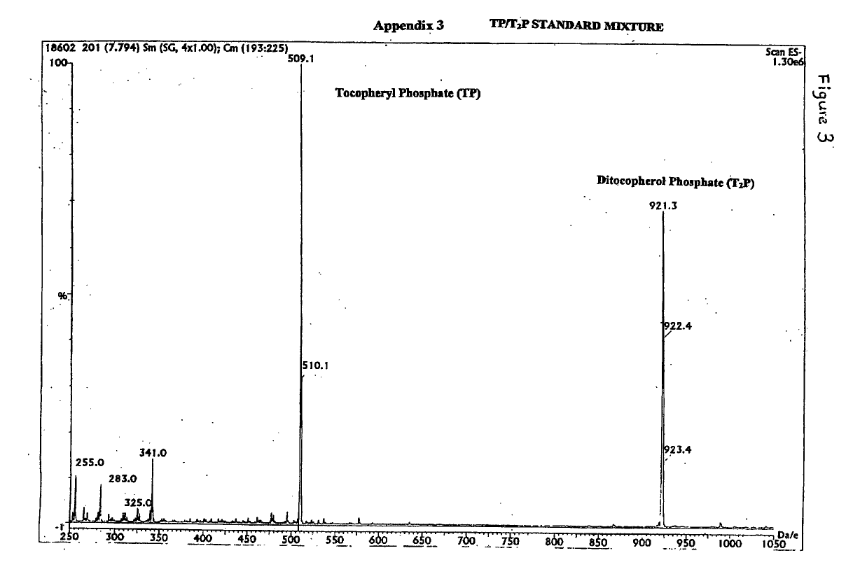

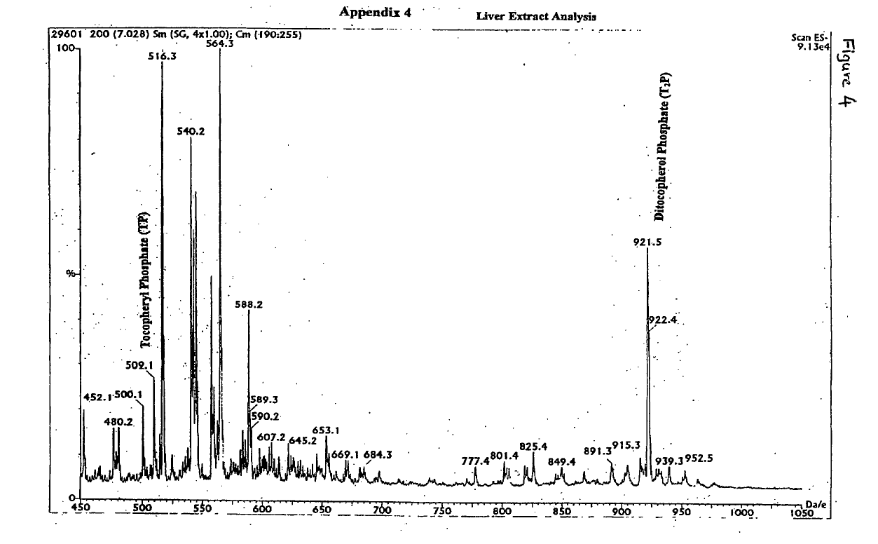

Although the stability of α-tocopheryl phosphate is such that vigorous conditions can be used to isolate it from tissue, but the detection of α-tocopheryl phosphate is extremely difficult and most common instrumentation is inadequate. Gas chromatography/mass spectrometry (GCMS) is the method of choice for α-tocopherol analysis, but this technique is not particularly suitable for analysis of α-tocopheryl phosphate. We were eventually able to detect α-tocopheryl phosphate by GCMS after derivatisation to dimethyl tocopheryl phosphate using diazomethane. This method was used to confirm the presence of endogenous α-tocopheryl phosphate in tissue samples, the endogenous material had the same retention time, molecular ion (m/z 538) and base peak (m/z 273) as the synthetic α-tocopheryl phosphate (see FIG. 2). This method was however found to be unsuitable for routine quantitative analysis. We examined a number of analytical techniques and determined that electrospray mass spectrometry (ESMS) was clearly the best method for the analysis of α-tocopheryl phosphate (see FIG. 3). α-Tocopheryl phosphate is readily ionisable and is soluble in water at high pH and in organic solvents at low pH; these are properties that are ideally suited to ESMS. With the addition of a suitable internal standard, levels of α-tocopheryl phosphate in tissues were routinely determined by ESMS (see FIG. 4).

Following on from the development of a suitable assay technique for α-tocopheryl phosphate, it was also necessary to develop an effective extraction protocol since α-tocopheryl phosphate could not be detected using the extraction methodology commonly employed for vitamin E determination. In the procedure we have developed, the tissue was homogenized in dichloromethane containing the internal standard, di-α-tocopheryl phosphate. The mixture was centrifuged and the dichloromethane layer was removed, leaving the majority of protein behind. The residue remaining after evaporation of the dichloromethane was then hydrolyzed with potassium hydroxide for 1 hour at room temperature. If the sample was not hydrolyzed then α-tocopheryl phosphate could not be detected, which suggests that it is associated with a protein or other some other compound; for example, the α-tocopheryl phosphate may be present as a glycerophosphate or similar ester, and that hydrolysis is required to liberate free α-tocopheryl phosphate. The hydrolysate was then washed with hexane to remove all of the organic soluble material. Free α-tocopherol was removed at this stage and could be readily analysed using standard techniques; α-tocopheryl phosphate was present as the potassium salt, and being water soluble was not extracted. Acidification of the aqueous layer converted the α-tocopheryl phosphate to the free acid, which was readily extracted into hexane.

Our findings that α-tocopheryl phosphate is resistant to hydrolysis and oxidation, coupled with the requirement to liberate free α-tocopheryl phosphate by alkaline hydrolysis during the extraction process, explains why the α-tocopherol present as α-tocopheryl phosphate has not previously been detected using the standard assays for vitamin E. Reported amounts of “total α-tocopherol” present in samples will consequently have always been underestimated.

Using this extraction protocol and ESMS analysis, the α-tocopheryl phosphate content of a range of animal and human tissues was measured. Table 1 presents the results of these analyses.

Naturally occurring α-tocopheryl phosphate has been detected in pig, guinea pig, chicken and rat liver, and in human, pig and guinea pig adipose tissue. Liver and adipose have been reported to be the two main storage sites for α-tocopherol and it is therefore not surprising that α-tocopheryl phosphate is also present in these tissues. Our results indicate that the α-tocopheryl phosphate makes up a significant proportion of the total α-tocopherol present.

TP has hitherto been considered to be unable to be deposited directly in tissues where it can remain as a storage form of tocopherol. However, the following example illustrates the detection of ingested TP in human and animal tissue.

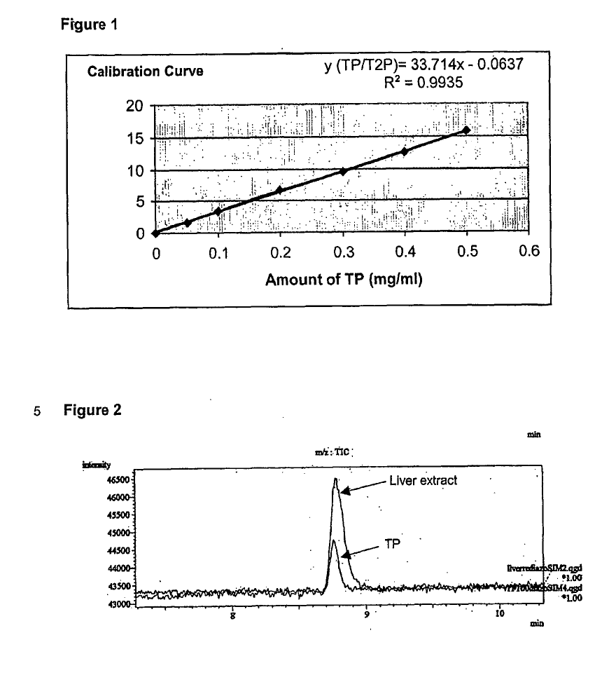

The level of TP was determined using ditocopheryl phosphate (T₂P) as the internal standard. T₂P is not a naturally occurring substance. A calibration curve was determined for TP vs T₂P (FIG. 1) using ES/MS. As can be seen from FIG. 1 the response is virtually linear.

An extraction method was developed which gave the maximum recovery of TP. The stability of TP to the extraction methodology was tested by subjecting pure TP to the extraction protocol, virtually 100% recovery was obtained. The extraction protocol is outlined below:

1. Weigh 1 g and homogenize in 10 ml dichloromethane.

2. Add 0.1 mg T₂P (1 mg/ml in 50% tetrahydrofuran) as internal standard.

3. Mix using homogenizer and centrifuge sample.

4. Remove and evaporate the dichloromethane.

5. Add 9 ml 2 M KOH (2M) and stir for 1 h at room temperature.

6. Add 10 ml hexane, shake and remove hexane (upper) layer.

7. Add 10 ml 2M HCl (2M) to the 9 ml KOH (2M) solution.

8. Add 10 ml hexane, shake and remove hexane layer.

9. Evaporate to dryness.

10. Analyse on ESMS, ESI (-ve mode)

ESMS Conditions

Sample was dissolved in 1 ml THF containing 1% NH₃, 20 μl was injected into the sample loop. The sample was eluted with THF:H₂O (9:1) 20 μl/min. MS analysis was conducted in ve ion mode with cone voltage of 40V.

Diazomethane Reaction with Liver Extract and TP Standard.

A solution of potassium hydroxide (5 g) in water (8 ml) and ethanol (10 ml) was preheated to 65° C. in a water bath. A solution of Diazald® (3.0 g, 13.8 mmol) in ether (40 ml) was slowly added to the potassium hydroxide solution and a distillate was distilled across. When the Diazald® addition was completed, 10 ml of ether was added and also distilled across. The final ether distillate contained approximately 420 mg (9.95 mmol) of diazomethane.

Half of this diazomethane solution was immediately added to a liver sample that had been extracted from 10 g of rat liver and pre-dissolved in THF and the other half of this diazomethane solution was added to 100 μg of TP (TP) standard that had been dissolved in ether. The two reaction mixtures were sealed and placed in a dark cupboard for approximately 30 minute, after which the solvent and diazomethane were removed from the liver extract and TP standard under reduced pressure. The methylated liver extract sample was dissolved in 100 μl of chloroform, as was the TP standard; 30 μl of this TP solution was then taken and re-dissolved in 1.5 ml of chloroform in order to mimic the concentration of TP in the liver extract. Both the liver extract and TP standard were then immediately analysed using gas chromatograph mass spectrometry (GC-MS).

GCMS Conditions

The methylated liver extract and TP standard were analysed using a Shimadsu GCMS-QP5000 Gas Chromatograph Mass Spectrometer incorporating a Shimadsu AOC-20i Auto Injector. One μl of sample was injected onto a 15 m SGE.BP1 capillary column, with a thickness of 0.25 μm and an internal diameter of 0.25 mm utilising a temperature program from 260° C. up to 300° C. ramping at 3° C. per minute. The injector and interface temperatures were both set at 300° C. The carrier gas used was helium, the column inlet pressure was set at 156 kPa and the total flow was 144 ml/min, thus presenting a column flow of 2.4 ml/min with a linear velocity of 86.5 cm/sec. The samples were run in split mode with a split ratio of 56:1.

The acquisition mode for the mass spectrometer was SIM (single ion monitoring) mode, with Channel 1-m/z=538.00 and Channel 2-m/z=273.00. The detector gain for this acquisition was set at 1.5 kVolts.

TP in Rat Liver

Non-treated rat livers were used to extract, detect and quantitate the amount of naturally occurring TP. Fresh livers from Sprague-dawley and Wistar rats (aged between 10 to 11 weeks old, weighing approximately 120-180 grams) were used for each extraction. The amount of naturally occurring TP in the rat livers was found to be 12.5 μg/g of liver (n=15).

TP has also been detected in human abdominal fat, Guinea Pig and rat adipose tissue, porcine and guinea pig liver of humans and animals which have not been administered TP. Table 1 outlines the quantity of TP detected in each organ.

The results demonstrate that the levels of tissue TP are of the same order of magnitude across the species. Interestingly, the phosphate form seems to be used in the same way, but not surprisingly, at different levels in different tissues depending upon the life span of the animal.

TABLE 1

| | Amount (μg/g tissue) |
|---|---|
| | TP detected |
| Species and Organ | using ESMS |
| Rat (male) Liver, n = 15 | 12 ± 4 |
| Rat (male) Adipose tissue, n = 5 | 18 ± 5 |
| Guinea Pig (male) Liver, n = 4 | 8 ± 3 |
| Guinea Pig (male) Adipose tissue, n = 4 | 10 ± 4 |
| Pig (7 month old-male) Liver, n = 3 | 18 ± 6 |
| Human Liver, n = 2 | 32 ± 2 |
| Human Adipose tissue, n = 3 | 25 ± 9 |
| Chicken (2 month old male) n = 14 | 36 ± 17 |

### Example 2

This example investigates whether tocopheryl phosphate levels in storage sites are increased after tocopheryl acetate and tocopheryl phosphate were administered.

The procedure used is summarized as follows:

(a) Administer a single dose of the compounds to male Sprague-Dawley rats (see table; oral gavage with an 18 g gavage needle and 1 ml syringe, or intravenously with a 26 g hypodermic needle and 1 ml syringe).

(b) Twenty-four hours after administrations the rat will be anaesthetized with Nembutal (60 mg/kg i.p.).

(c) Once the rats were under deep anaesthesia a sample of blood was taken from the tail vein, and the femoral vein exposed and injected with 500 units of heparin. The abdominal cavity will be opened and the rat perfused with saline. The liver, heart and epidydimal fat pad removed and frozen in liquid nitrogen. Hind-leg muscle and brain will also be collected and frozen.

TABLE 2
Treatments

| | | Number of |
|---|---|---|
| Compound | Dose (mg/kg) | Rats |
| TP (IV) | 10 | 3 |
| TP (IV) | 30 | 3 |
| TP | 10 | 3 |
| TP | 30 | 3 |
| Tocopheryl acetate | 10 | 3 |
| Tocopheryl acetate | 30 | 3 |
| Tocopheryl acetate | 100 | 3 |
| Control (0.3 ml water)(IV) | 0 | 3 |
| Control (0.3 ml corn oil) | 0 | 3 |

Livers were be extracted according to the method below. The extracts will be analyzed and quantitated for TP (μg) content by ESMS. Any tissue samples left over at the end of the study was kept frozen at −80° C.

Liver Extraction

One g liver was homogenized in 10 ml dichloromethane (analytical grade). Add 0.1 mg tocopheryl diphosphate (internal standard). Homogenize sample for 2 min, centrifuge and remove upper layer and evaporate under nitrogen. Add 9 ml KOH (2M) and stir for 1 hr at room temperature (or 20 min at 80 C). Add 10 ml hexane, shake and remove upper layer. Add 10 ml HCl (2M) and shake. Add 10 ml hexane to the solution and shake and remove upper layer. Evaporate top layer to dryness.

Electrospray Analysis

Add 1 ml tetrahydrofuran (THF) and 20 ul of 25% Ammonia to sample and analyse.

Results

TABLE 3
Treatment dose of TP (mg/kg) vs. liver TP (ug/gm)

| | Control | | | | |
|---|---|---|---|---|---|
| | (Oral & | | | | |
| Treatment | i.v.) | 3 mg/kg | 10 mg/kg | 30 mg/kg | 100 mg/kg |
| TP i.v. | 11.95 ug | 17 ug | 24 ug | 28 ug | 27 ug |
| | | (61%) | (86%) | (100%) | |
| TP oral | | | 19.18 ug | 18.35 ug | |
| | | | (68%) | (65%) | |
| TA oral | 8.0 ug | | 12.81 ug | 13.71 ug | 14 ug |
| | | | (46%) | (49%) | (50%) |

TP was administered by IV to provide a value for absolute bioavailability. The amounts in brackets are percentages when compared with the IV value.

Conclusion

The above results in table 3 clearly demonstrate that:

(a) absorption of a tocopherol analogue can improve tissue levels of TP within a 24 hour period; from which it can be inferred that the acetate group has been biologically removed and replaced by a phosphate group

(b) TP has higher bioavailability than tocopheryl acetate because the transformation to phosphate is not required

This means that the higher bioavailability of TP can quickly increase tissue concentrations of the electron transfer agent to therapeutically efficacious levels.

### Example 3

This example investigates whether tocopheryl phosphate exists in commonly consumed food products in a normal diet using analytical methods outlined in example 1.

Table 4 shows the results of TP analysis in chicken fat, muscle and eggs.

TABLE 4
Concentration of TP in chicken fat, muscle and eggs as determined by
ESMS

| | Ratio of | | |
|---|---|---|---|
| | TP/T2P | | |
| Chicken Number & | (SIR | | |
| Tissue | plots) | [TP] μg/g tissue | Mean ± SD |
| 1. muscle | 0.3193 | 13 | 9 ± 5 |
| 2. muscle | 0.1325 | 3 | |
| 3. muscle | 0.2560 | 10 | |
| Eggwhite black-x | 0.0633 | Below limit of detection | |
| Eggwhite white-x | 0.0419 | Below limit of detection | |
| Eggwhite white-x | 0.0512 | Below limit of detection | |
| Eggwhite brown-x | 0.0361 | Below limit of detection | |
| Eggwhite brown-x | 0.0633 | Below limit of detection | |
| Fat | 5.724 | 301 | 310 ± 35 |
| Fat | 5.333 | 280 | |
| Fat | 6.64 | 349 | |
| Yolk black-x | 0.9578 | 47 | 40 ± 9 |
| Yolk white-x | 0.6747 | 32 | |
| Yolk white-x | 0.6145 | 29 | |
| Yolk brown-x | 0.9548 | 47 | |
| Yolk brown-x | 0.9608 | 47 | |

Clearly large amounts of TP were detected in a variety of commonly consumed chicken products. This means that TP is consumed in normal diets containing poultry meats, fat and eggs, suggesting the compound is associated with a long history of safe use. Importantly however, the levels required for disease intervention would likely be inadequate from these sources and additional doses of TP are required.

The word ‘comprising’ and forms of the word ‘comprising’ as used in this description and in the claims does not limit the invention claimed to exclude any variants or additions.

Modifications and improvements to the invention will be readily apparent to those skilled in the art. Such modifications and improvements are intended to be within the scope of this invention.

1. A method for increasing levels of a storage form of a vitamin selected from the group consisting of tocopherol, retinol, vitamin K1 and mixtures thereof in a target tissue of a subject, the method comprising administering to the subject an effective amount of a phosphate derivative of the vitamin so as to cause an accumulation of stored vitamin in the target tissue.

2. A method according to claim 1 wherein the vitamin is tocopherol.

3. A method according to claim 1 or 2 wherein the subject is a human.

4. A method according to any one of claims 1 to 3 wherein the target tissue is selected from the group consisting of liver, skin and adipose tissue.

5. A method according to any one of claims 1 to 3 wherein the target tissue is brain.

6. A method according to any one of claims 1 to 5 wherein the increased levels of the stored vitamin result in providing therapeutic levels of the vitamin to the animal.

7. A method according to claim 6 wherein the therapeutic levels of the vitamin result in alleviation or treatment of inflammatory disease.

8. A method according to claim 7 wherein the inflammatory disease is selected from the group consisting of coronary diseases, artherosclerosis and diabetes.

9. A method according to claim 6 wherein the therapeutic levels of the vitamin result in alleviation or treatment of cancer.

10. A method according to claim 6 wherein the therapeutic levels of the vitamin result in alleviation or treatment of problems of foetal development.

11. A method according to claim 6 wherein the therapeutic levels of the vitamin are at least about 50% more than levels usually stored in healthy or normal tissue.

12. A method according to any one of claims 1 to 11 wherein the effective amount of the phosphate derivative of the vitamin is at least about 3 mg/kg body mass of the animal.

13. A method according to claim 12 wherein the effective amount of the phosphate derivative of the vitamin is in the range of 3 to 50 mg/kg body mass of the animal.

14. A method according to claim 13 wherein the effective amount of the phosphate derivative of the vitamin is at least 10 mg/kg body mass of the animal.

15. A method according to any one of claims 1 to 14 wherein the phosphate derivative is a phosphatide.

16. A method according to any one of claims 1 to 15 wherein the phosphate derivative is a complex of the phosphate derivative of the vitamin.

17. A method for alleviating or treating a subject suffering a condition responsive to a vitamin treatment, the method comprising administering to a subject in need of such treatment an amount of a phosphate derivative of the vitamin selected from the group consisting of tocopherol, retinol, vitamin K1 and mixtures thereof effective to cause an accumulation of a therapeutic amount of tocopherol, retinol vitamin K1 or a mixture thereof in a tissue of the subject.

18. A method according to claim 17 wherein the vitamin is tocopherol.

19. A method according to claim 17 or 18 wherein the subject is a human.

20. A method according to any one of claims 17 to 19 wherein the tissue is liver.

21. A method according to any one of claims 17 to 20 wherein the phosphate derivative is a phosphatide.

22. A method according to any one of claims 17 to 21 wherein the phosphate derivative is a complex of the phosphate derivative of the vitamin.

23. Use of an effective amount of a phosphate derivative of a vitamin selected from the group consisting of tocopherol, retinol, vitamin K1 and mixtures thereof in the manufacture of a supplement for causing an accumulation of stored vitamin in the target tissue of an animal.

24. Use of an effective amount of a phosphate derivative of a vitamin selected from the group consisting of tocopherol, retinol, vitamin K1 and mixtures thereof in the manufacture of a medicament for alleviating or treating a subject suffering a condition responsive to a vitamin treatment.
